(19) United States
(12) Patent Application Publication
(10) Pub. No.: US 2026/0079116 A1
(43) Pub. Date: Mar. 19, 2026

(54) MEDICATION CONTAINER INSPECTION DEVICE

(71) Applicant: LUO AUTOMATION B.V., DELFT (NL)

(72) Inventors: Joren Ide VAN DER HORST, DELFT (NL); Jim KIEFT, DELFT (NL); Joseph Roeland HENDRIKS, DELFT (NL)

(21) Appl. No.: 19/109,156

(22) PCT Filed: Sep. 7, 2023
(86) PCT No.: PCT/NL2023/050459

§ 371 Date: Mar. 6, 2025

(30) Foreign Application Priority Data
Sep. 7, 2022 (NL) 2032976

(51) Int. Cl. G01N 21/90; A61M 5/178

(52) U.S. Cl. CPC G01N 21/9009; A61M 5/178

(57) ABSTRACT

The invention relates to a medication container inspection device (1) for inspecting one or more medication containers (2), comprising:—an inspection station (4) with an inspection system (5) comprising an inspection apparatus (6), conured for inspecting a single medication container for container defects, and a manipulator to entangle or agitate the single medication container to increase the visibility of container defects while being inspected by the inspection apparatus,—a transport system (10) for automatically transporting one or more medication containers to the inspection station (4), wherein the one or more medication containers (2) can be fed to the transport system (10) with an infeed (9), wherein the inspection system (5) is configured to perform multiple inspecting steps in the inspection station (4).

The present invention relates to a medication container inspection device for inspecting a single medication container, in particular for inspecting a single medication containers for container defects, such as contamination of contents of the medication container, closure integrity of the medication container, or cosmetic defects, as well as a medication container inspection assembly, comprising such a medication container inspection device, a method for inspecting a single medication container using such a medication container inspection device or such a medication container inspection assembly, and an infeed for feeding one or more medication containers to such a medication container inspection device.

### PRIOR ART

Container inspection devices for inspecting containers, in particular for inspecting containers for container defects, are known in the art.

US 2021/0293725 A1 for instance discloses an inspection system for the inspection of container units that combines and integrates a holding assembly for multiple containers integrating servo-controlled rotation of the units, transport and positioning of the containers that simulate human handling, and camera stations employing automated vision inspection. The system performs horizontal inspection for particulate and any other container defect that promotes particulate to better locate within the inspection area of the cameras. Inspection sequences and product recipes combine the typical manual inspection agitation with automated inspection rotational techniques to optimize detection. The system allows for semi-automatic operation with the operator at the front of the station feeding and out-feeding material manually or fully automated with a conveyance system feeding and out-feeding material from the back of the station.

Furthermore, commercially available container inspection devices are produced by for example Stevanato, Körber, Sterisys, Gronigner, Brevetti or Syntagon, to name a few. Such devices are primarily configured for inspecting medication containers.

However, a problem with the above prior art container inspection devices is that such container inspection devices are generally geared towards relatively high throughputs (in the order of 10000's of containers per hour), implying relatively large devices, requiring lots of space and, from a financial perspective, requiring major financial investments for acquiring such devices. Thus, such prior art devices are not suitable for use by relatively “small” users, such as (when the container comprises a medication) pharmacies, companies or hospitals seeking to inspect only relatively small quantities or batches of containers for contamination or closure integrity (for instance at a throughput of several 100's of containers per hour). Of course, manual inspection of relatively small batches is relatively cheap, but also brings its own set of problems, such as low throughput (in the order of 300-600 of containers per hour) and an increased risk of (human) error. Summarizing, the prior art inspection devices or inspection methods lack “scalability” with respect to throughput and/or batch size.

Another major disadvantage of such relatively large prior art container inspection devices is that e.g. “retooling” or, in general, adapting the device for inspection of a different type of container often requires several hours of work, again making the prior art devices relatively unsuitable for inspection of relatively small batches.

### Object of the Invention

An object of the present invention is thus to provide a medication container inspection device, in particular for inspecting medication containers for container defects, that takes up relatively little space.

A further object of the invention is to provide a medication container inspection device that is suitable for the processing of relatively small batches of medication containers, at a rate/throughput of for instance 100's or 1000's of medication containers per hour.

### SUMMARY OF THE INVENTION

According to the present invention, a medication container inspection device for inspecting one or more medication containers is provided, comprising:
an inspection station with an inspection system comprising an inspection apparatus, configured for inspecting a single medication container for container defects, and a manipulator to entangle or agitate the single medication container to increase the visibility of container defects while being inspected by the inspection apparatus,
a transport system for automatically transporting one or more medication containers, such as a single medication container, to the inspection station, wherein the one or more medication containers can be fed to the transport system with an infeed,
wherein the inspection system is configured to perform multiple inspecting steps in the inspection station.

The Applicant has found that the above container inspection device achieves high quality inspection of relatively small batches, at relatively small throughputs. Furthermore, such a container inspection device takes up relatively little space and is interesting to acquire from a financial perspective when relatively small batches need to be inspected. Preferably, the transport system is a system separate from the infeed.

US 2017/0345141 A1 discloses a method and apparatus for detection of foam in specimen containers. US 2017/0345141 A1 is particularly aimed at the detection of microorganisms in biological fluids, especially blood, in specimen containers (see [0002] of US 2017/0345141 A1) with the main aim of preventing blood poisoning (again see paragraph [0002] of US 2017/0345141 A1). Inspection of medication is not mentioned anywhere in US 2017/0345141 A1.

Furthermore, the robotic head 656, as e.g. disclosed in FIG. 5A of US 2017/0345141 A1, cannot be regarded as a manipulator to entangle or agitate the container to increase the visibility of container defects while the container is being inspected by the inspection apparatus. The robotic head 656 has a completely different function within the device of US 2017/0345141 A1: the robotic head 656 is configured for storing a container in a rack 600 in an incubation chamber 620, see for example paragraph [0124] of US 2017/0345141 A1.

As mentioned before, US 2021/0293725 A1 is configured for inspecting several syringes at a time with US 2021/0293725 A1 thereto disclosing feeding and holding several syringes in the inspection room at a time, by means of a robot arm supporting the several syringes.

In the context of the present patent application, “container defects” may for instance refer to contamination of contents of the container, closure integrity defects of the container, or cosmetic defects. Furthermore, an “inspection station” is the physical or imaginary space where the inspection system operates. The inspection system comprises the manipulator to entangle or agitate the container, and the “inspection apparatus”. The inspection apparatus is a system to record information to inspect the single medication container without the medication container leaving the field of view or in a more general sense the “region of interest”, as the skilled person will understand. Recording information may involve the use of near-ultraviolet (NUV), visible, near-infrared (NIR), mid-infrared (MIR) and/or far-infrared (FIR) wavelengths/frequencies. In the inspection station multiple inspection steps can be performed on a single container in consecutive order. An inspection step is (1) a specific measurement performed by the inspection system, comprising an action or position of the manipulator, wherein the action or position may be executed before, during or after the recording, or (2) a specific setting of the inspection apparatus. A medication container comprises a medication, also called medicament, medicine, pharmaceutical drug, medicinal drug or simply drug, i.e. a drug used to diagnose, cure, treat, or prevent disease. A medication may comprise a medicine or a chemical compound used to treat or cure illness. A medication may also be regarded as a substance used in treating a disease or relieving pain. Example medications include antipyretics, analgesics, antimalarial drugs, antibiotics, antiseptics, mood stabilizers, hormone replacements, oral contraceptives, stimulants, tranquilizers and statins. Such a medication may comprise a solid medication, e.g. including a freeze-dried product, a fluid medication, or a mix thereof.

An embodiment relates to an aforementioned container inspection device, comprising:
a housing, wherein the inspection station is arranged in the housing, with an infeed opening, or multiple infeed openings, arranged at a side of the housing, for allowing an infeed to feed one or more medication containers to the infeed opening, wherein the transport system is arranged for automatically transporting one or more medication containers to the inspection station from the infeed opening or infeed openings. Thus, a container can be conveniently fed to the infeed opening, whereafter the container is automatically transported to the inspection station by the transport system. “Side” may comprises a bottom side, top side, horizontal side, et cetera, basically any outer surface of the housing.

An embodiment relates to an aforementioned container inspection device, wherein the inspection system is configured for engaging the single medication container at a plurality of engagement positions on the single medication container, such as at least two engagement positions, for instance at opposite ends of the container. Thus, the container can be manipulated by the manipulator and viewed from many different positions and angles, while the container is firmly held at the engagement positions. The container may e.g. be moved (“translation”) or turned around a longitudinal or transversal axis of the container (“rotation”).

An embodiment relates to an aforementioned container inspection device, wherein the inspection system is configured to perform all of the multiple inspecting steps in the inspection station. By doing so, the whole inspection process can be completed inside the inspection station, without having to transport the container to further inspection stations.

An embodiment relates to an aforementioned container inspection device, wherein the inspection apparatus comprises one or more viewing devices for viewing the single medication container in the inspection station. Such viewing devices may for instance comprise cameras or the like.

An embodiment relates to an aforementioned container inspection device, wherein the inspection system comprises one or more illumination devices for illuminating the single medication container in the inspection station when the single medication container is viewed by the one or more viewing devices.

An embodiment relates to an aforementioned container inspection device, wherein the side of the housing comprising the infeed opening is the, during use, top side of the housing. Thus, the infeed opening is easily accessible and gravity may advantageously be used to transport the container to the inspection station. “Top side” therein means the top surface of the housing during normal use.

An embodiment relates to an aforementioned container inspection device, wherein the infeed opening is arranged vertically above the inspection station, such that the transport system can transport the one or more medication containers vertically from the infeed opening to the inspection station. As mentioned in the previous paragraph, gravity may thus be used to transport the container into the inspection station with the transport system, which may for instance comprise a lift device or the like, in particular a powered lift device.

An embodiment relates to an aforementioned container inspection device, wherein the manipulator doubles as the transport system for automatically transporting the one or more medication containers to the inspection station. Mutatis mutandis, the transport system may also double as the manipulator to entangle or agitate the one or more medication containers.

An embodiment relates to an aforementioned container inspection device, wherein the single medication container is at least partly transparent, such that the contents of the single medication container can be viewed from outside the container.

An embodiment relates to an aforementioned container inspection device, wherein the single medication container comprises a fluid and/or a solid, i.e. a fluid and/or solid used for medical purposes.

An embodiment relates to an aforementioned container inspection device, wherein the single medication container comprises a syringe, vial or ampoule. The skilled person will understand that the container may also comprise bottles or the like.

As mentioned before, the container inspection device as disclosed in the present patent application is particularly useful for the pharmaceutical profession, wherein inspection of medication, for instance comprised by syringes, vials or ampoules (or bottles), is required.

An embodiment relates to an aforementioned container inspection device, comprising:
multiple inspection stations each with an inspection system comprising an inspection apparatus, configured for inspecting a single medication container for container defects, and a manipulator to entangle or agitate the single medication container to increase the visibility of container defects while being inspected by the inspection apparatus, wherein at least one of the inspection stations is configured to perform multiple inspecting steps in the inspection station, and
a transport system for automatically transporting one or more medication containers to each of the inspection stations, wherein the one or more medication containers can be fed to the transport system with an infeed.

The above container inspection device with multiple inspection stations allows for a modular design of the container inspection device. For instance, when increased throughput is required (e.g. X containers/hour versus 2 * X containers/hour), additional inspection stations may be conveniently added. With one inspection station the Applicant has for instance achieved a throughput of 300 containers/hour, whereas with two inspection stations a throughput of 600 containers/hour has been achieved. Three, four or more inspection stations are of course also conceivable.

Another aspect of the invention concerns a medication container inspection assembly, comprising:
an aforementioned medication container inspection device; and
an infeed arranged for feeding one or more medication containers to the transport system.

Preferably, the infeed is an automated infeed that automatically and consecutively supplies a container to the transport system. Together with the automated transport system, the container can then be fully automatically transported from “outside” the container inspection device into the inspection station (without requiring manual intervention from a human operator).

An embodiment relates to an aforementioned container inspection assembly, wherein the infeed is arranged at a side of the housing for feeding the one or more medication containers to the infeed opening.

An embodiment relates to an aforementioned container inspection assembly, wherein the infeed holds vials, ampoules, syringes or a nest of syringes.

Preferably, the infeed is configured for feeding the syringes, vials or ampoules to the infeed opening or, if applicable, the syringes to the infeed opening from the nest. In particular syringes are often stored or handled in a nest, i.e. a tray holding several tens of syringes.

An embodiment relates to an aforementioned container inspection assembly, wherein the nest comprises syringes with injection ends, for instance comprising syringe caps, wherein the syringes are arranged in the nest with the injection ends upwards, wherein the syringes are fed to the transport system with the injection ends upwards.

An embodiment relates to an aforementioned container inspection assembly, wherein the syringes are fed to the infeed opening with the injection ends upwards. A major advantage of supplying the syringes to the transport system or the infeed opening with the injection ends upwards is that so-called “de-nesting” is not required, leading to high throughputs and less complex design. The syringes may basically “fall”, preferably one by one, into the transport system to be transported to the inspection station (preferably aided by gravity).

An embodiment relates to an aforementioned container inspection assembly, wherein the transport system is arranged for automatically transporting syringes from the infeed opening to the inspection station with the injection ends upwards. Thus, the transport system does not have to rotate syringes with the injection ends upwards before transporting such syringes into the inspection station. As the skilled person will understand, inspection of such syringes preferably also takes place with the syringes being oriented with the injection ends upwards. It is even conceivable that the infeed opening is used as an outfeed opening, wherein, after inspection, the transport system transports the syringes from the inspection station towards the outfeed opening in order to be placed back in the nest.

An embodiment relates to an aforementioned container inspection assembly, comprising an outfeed for transporting a single medication container away from the inspection station or, when the container inspection device comprises multiple inspection stations, medication containers away from each of the multiple inspection stations. As mentioned in the previous paragraph, the infeed opening may function as an outfeed opening and the infeed then essentially functions as an outfeed, wherein the transport system is used to transport the container to/from the inspection station from/to the infeed/outfeed opening. Of course, the outfeed opening may also be a separate opening in (a side of) the housing, and the outfeed may be a separate module or device.

Another aspect of the invention concerns a method for inspecting a single medication container using an aforementioned medication container inspection device or an aforementioned medication container inspection assembly, comprising:
automatically transporting one or more medication containers to the inspection station with the transport system,
inspecting a single medication container for container defects with the inspection system comprising the inspection apparatus, wherein the inspection system performs multiple inspecting steps in the inspection station, and entangling or agitating the single medication container to increase the visibility of container defects while being inspected by the inspection apparatus.

Another aspect of the invention relates to an infeed for feeding a one or more medication containers to the transport system of an aforementioned medication container inspection device, for instance configured as an automated infeed for automatically supplying one or more medication containers to, for example, the infeed opening.

### BRIEF DESCRIPTION OF THE DRAWINGS

The invention will be explained in more detail below, with reference to illustrative embodiments shown in the drawings. Therein:

FIG. 1 shows a first, schematic example embodiment of a container inspection assembly with a container inspection device according to the present invention, with a single inspection station;

FIG. 2 shows a second, schematic example embodiment of a container inspection assembly with a container inspection device according to the present invention, with two inspection stations;

FIG. 3 shows an example embodiment of an inspection system for use in a container inspection device according to the present invention;

FIG. 4 shows a perspective view of an example embodiment of a container inspection assembly with a container inspection device according to the present invention, with two inspection stations, wherein the container inspection assembly is in a closed state;

FIG. 5 shows a perspective view of an example embodiment of a container inspection assembly with a container inspection device according to the present invention, with two inspection stations, wherein the container inspection assembly is in an open state; and

FIG. 6 shows a schematic view of an example embodiment of a method for feeding a syringe from a syringe nest to the transport system via an infeed opening with the injection end upwards.

### DETAILED DESCRIPTION

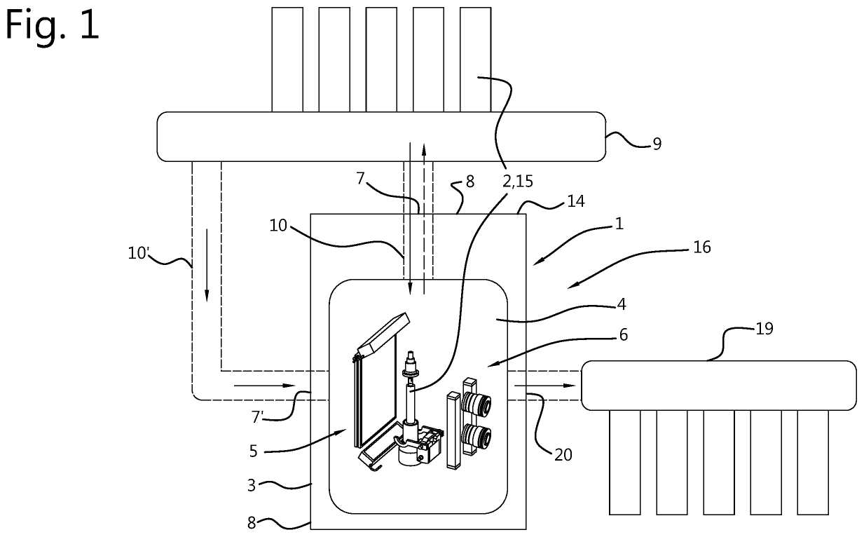

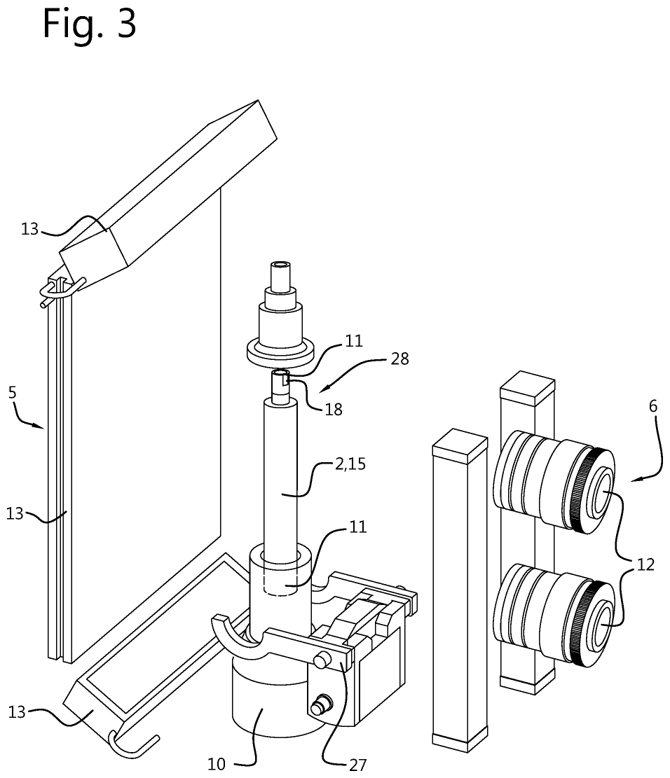

As mentioned in the foregoing, FIG. 1 shows a first, schematic example embodiment of a medication container inspection assembly 16 with a medication container inspection device 1 according to the present invention, with a single inspection station 4. The inspection station 4 comprises an inspection system 5 with an inspection apparatus 6, configured for inspecting a single medication container 2 for container defects, and a manipulator (please see FIG. 3, reference numeral 27) to entangle or agitate the single medication container 2 to increase the visibility of container defects while being inspected by the inspection apparatus 6. A transport system 10, 10′ is provided (shown in two alternative locations) for automatically transporting one or more medication containers to the inspection station 4, e.g. by moving a medication container 2 in a longitudinal or transversal direction of the medication container 2, for instance in a vertical direction or a horizontal direction, preferably at a transport speed of 2-20 cm/s, more preferably 5-10 cm/s. The medication container 2 can be fed to the transport system 10 with an infeed 9. The inspection system 5 is configured to perform multiple inspecting steps in the inspection station 4. As mentioned before, inspecting may comprise recording information involving the use of e.g. near-ultraviolet (NUV), visible, near-infrared (NIR), mid-infrared (MIR) and/or far-infrared (FIR) wavelengths/frequencies.

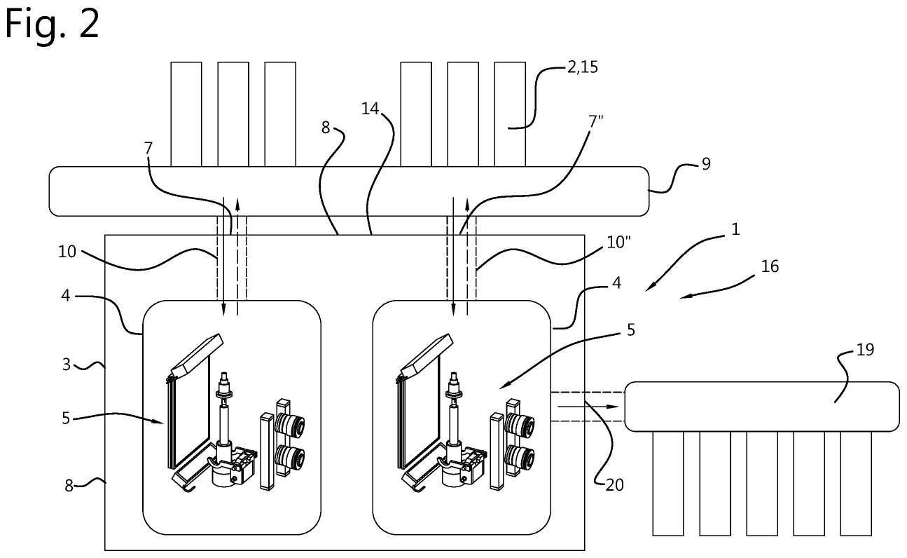

The medication container inspection device 1 for inspecting one or more medication containers 2 as shown in FIG. 1 comprises a housing 3, for instance having a cubic or “brick” shape. The dimensions of the housing 3 depend on the application, required throughput, the positioning of infeeds and outfeeds, et cetera. Purely by means of example, the dimensions of the housing 3 may amount to 0.25-1.0 m (width), 0.5-1.0 m (height) and 0.25-1.0 m (depth), i.e. for a medication container inspection device 1 with a single inspection station 4. A container inspection device 1 with two inspection stations 4 may have a width of for instance 0.5-2.0 m (height and depth may be similar to the container inspection device 1 with a single inspection station 4). The housing 3 may be made of plastic, metal, et cetera. An inspection station 4 is arranged in the housing 3, with an inspection system 5 comprising an inspection apparatus 6, configured for inspecting a single medication container 2 for container defects, such as contamination of contents of the medication container 2, closure integrity defects of the container, or cosmetic defects. The inspection system 5 is configured for entangling or agitating the single medication container 2 with the manipulator while being inspected by the inspection apparatus 6, to impart movement on particles present in the single medication container 2, e.g. by means of rotating or translating the single medication container 2 along, or around, one, two or three axes. Preferably, the medication container 2 is rotated around in particular the longitudinal axis of the medication container 2 at a speed of between 500 and 3.000 rpm, such as between 1.000 and 2.000 rpm, to impart movement on the particles in order to make such particles visible. An infeed opening 7 is arranged at a side 8 of the housing 3 for allowing an infeed 9 arranged at the side 8 of the housing 3 to feed one or more medication containers 2 to the infeed opening 7. Preferably, the infeed opening 7 is configured for allowing a single medication container 2 at a time to pass through the infeed opening 7. The infeed opening may be circular, although other shapes are also conceivable. The side 8 of the housing 3 comprising the infeed opening 7 may be the, during use, top side 14 of the housing 3, as shown in FIG. 1. A transport system 10 is arranged in the housing 3, for automatically transporting the one or more medication containers 2 from the infeed opening 7 to the inspection station 4. Alternatively, or in addition to, the infeed opening 7 and transport system 10, an infeed opening 7′ and transport system 10′ may be provided, as shown in FIG. 1, for feeding one or more medication containers 2 from a side 8 of the housing 3 not being the top side 14 or a bottom side. The transport direction of the container 2 is indicated with arrows. The medication container inspection assembly 16 as shown in FIG. 1 also comprises an outfeed 19 for transporting a medication container 2 away from the inspection station 4 or, as shown in FIG. 2, medication containers 2 away from each of the multiple inspection stations 4 through an outfeed opening 20. In some embodiments, the infeed opening 7 may also function as an outfeed opening, i.e. the medication container 2 leaves the housing 3 after inspection through the same opening as through which it was fed into the housing 3 (as indicated by the dashed arrow).

FIG. 1 further shows that the infeed opening 7 may be arranged vertically above the inspection station 4, such that the transport system 10 can transport the one or more medication containers 2 vertically, i.e. downwards, from the infeed opening 7 to the inspection station 4.

The medication container 2 may be at least partly transparent, such that the contents of the container 2 can be viewed from outside the medication container 2. The medication container 2 may further comprise a fluid and/or a solid. The container 2 may be formed by a syringe 15, vial, bottle or ampoule and the container 2 comprises a medication.

FIG. 2 shows a second, schematic example embodiment of a medication container inspection assembly 16 with a medication container inspection device 1 according to the present invention, with two inspection stations 4, i.e. multiple inspection stations 4 are arranged in the housing 3, such as two, as shown in FIG. 2. Each inspection station 4 comprises an inspection system 5 with an inspection apparatus 6, configured for inspecting a single medication container 2 for container defects. Each inspection system 5 is configured for agitating the single medication container 2 with the manipulator while being inspected inside the inspection station 4, to impart movement on particles present in the container 2 or change the viewing position or angle. The transport system 10, 10′ in the housing 3 is arranged for automatically transporting medication containers 2 fed to the infeed opening 7, 7′ to each of the inspection stations 4. After inspection, the medication container(s) 2 may be moved out of the housing 3 by means of the outfeed 19 and outfeed opening 20. Again, the medication container(s) 2 may also be expelled from the infeed opening(s) 7, 7″.

FIG. 3 shows an example embodiment of an inspection system 5 for use in a medication container inspection device 1 according to the present invention, such as medication container inspection devices 1 shown in FIGS. 1-2 and 4-5. The inspection system 5 is preferably configured for engaging or supporting the single medication container 2 at an engagement position 11 or a plurality of engagement positions 11 on the single medication container 2, such as engagement positions 11 at opposite ends of the medication container 2, e.g. a lower end and/or an upper end of the single medication container 2, for instance in the form of a syringe 15 (wherein an upper end of the syringe 15 is an injection end 28 with a syringe cap 18). The inspection apparatus 6 may comprise one or more, such as two, viewing devices 12, for instance in the form of cameras, for viewing the container 2 in the inspection station 4. The inspection system 5 may comprise one or more illumination devices 13 for illuminating the single medication container 2 in the inspection station 4 when the single medication container is viewed by the one or more viewing devices 12, for instance a backlight with a pair of (angled) top/bottom lights, as shown in FIG. 3. Specific light filters can be used to prevent the different illumination devices from interfering with each other or disturbing measurements that are executed with other illumination devices. Preferably, the one or more viewing devices 12 are arranged on one side of the single medication container 2 and the one or more illumination devices 13 on an opposite side of the single medication container 2. The manipulator 27 is also shown. The inspecting comprises inspecting steps, wherein the inspection system 5 is configured to perform multiple, such as all of the inspecting steps in the inspection station 4. At the lower engagement position 11, as shown in FIG. 2, the single medication container 2 may be held by an engagement device which may double as a transport system 10. The engagement device 10 may for instance be formed by a mechanically or electrically actuated “lift” configured to receive the one or more medication containers 2 from the infeed opening 7 in order to transport the one or more medication containers into the inspection station 4. The engagement device 10 may also be used to transport the single medication container 2 back to the infeed opening 7 after inspection, as explained in the foregoing. The engagement device may also be part of the manipulator 27.

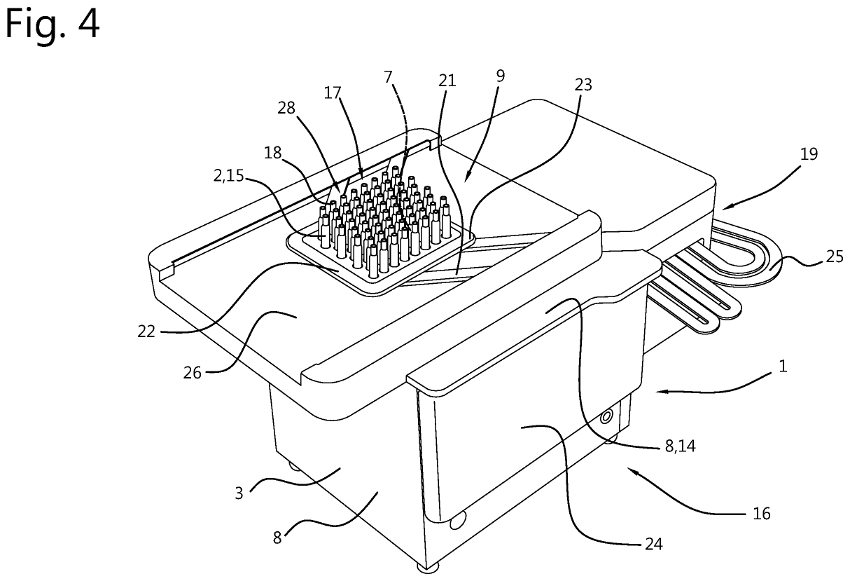

FIG. 4 shows a perspective view of an example embodiment of a medication container inspection assembly 16 with a medication container inspection device 1 according to the present invention, with two inspection stations, wherein the medication container inspection assembly 16 is in a closed state, i.e. a lid 24 is provided at a front side of the medication container inspection device 1 and the lid 24 is closed. As shown in FIG. 4, the infeed 9 holds a nest 17 of syringes 15 (although the infeed 19 may also hold vials, bottles or ampoules), wherein the infeed 19 is configured for feeding the syringes 15 to the infeed opening 7 from the nest 17. As shown in FIG. 2, the nest 17 comprises syringes 15 with injection ends 28, for instance comprising syringe caps 18, wherein the syringes 15 are arranged in the nest 17 with the injection ends 28 upwards. The syringes 15 are fed to the infeed opening 7 with the injection ends 28 upwards. The transport system 10 is arranged for automatically transporting syringes 15 from the infeed opening 7 to the inspection station 4 with the injection ends 28 upwards. The nest 17 may for instance have an 8×8 configuration (wherein rows of syringes 15 are slightly offset), as shown in FIG. 4, but other configurations are also conceivable. The infeed 9 may comprise a nest platform 22 for holding the nest 17. The nest platform 22 may be moveably connected to an infeed table 26 with mechanical links 21 and joints 23, such as two pairs of opposing mechanical links 21. The infeed 9 preferably is configured to move the nest platform 22 over a top surface of the infeed table 26, in order to automatically position an individual syringe 15 to be inspected over the infeed opening 7.

The outfeed 19 may for instance comprise a rail 25 or the like to transport medication containers 2 away from the container inspection device 1 after inspection.

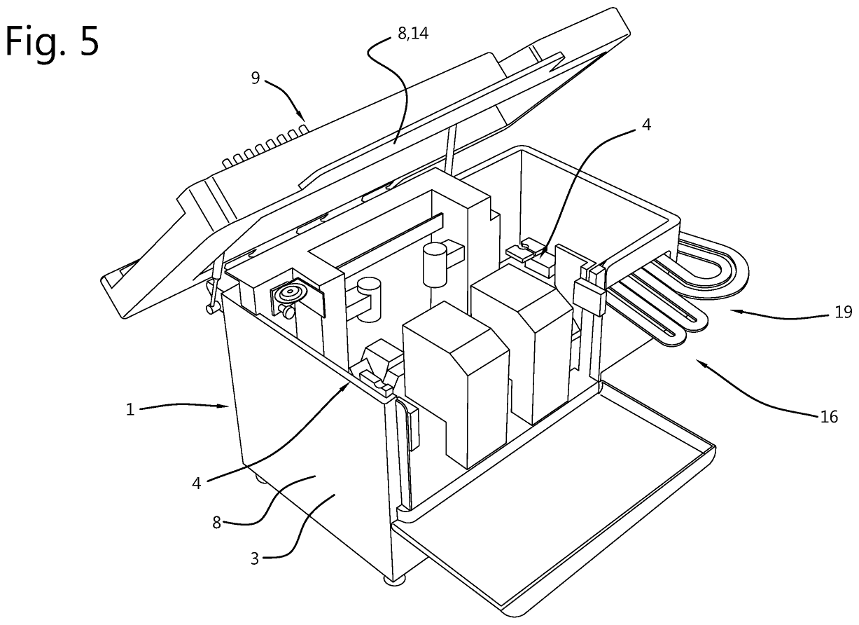

FIG. 5 shows a perspective view of an example embodiment of a medication container inspection assembly 16 with a medication container inspection device 1 according to the present invention, with two inspection stations 4, wherein the medication container inspection assembly 16 is in an open state, i.e. the lid 24 provided at a front side of the medication container inspection device 1 is now open to provide a better view of the interior of the medication container inspection device 1.

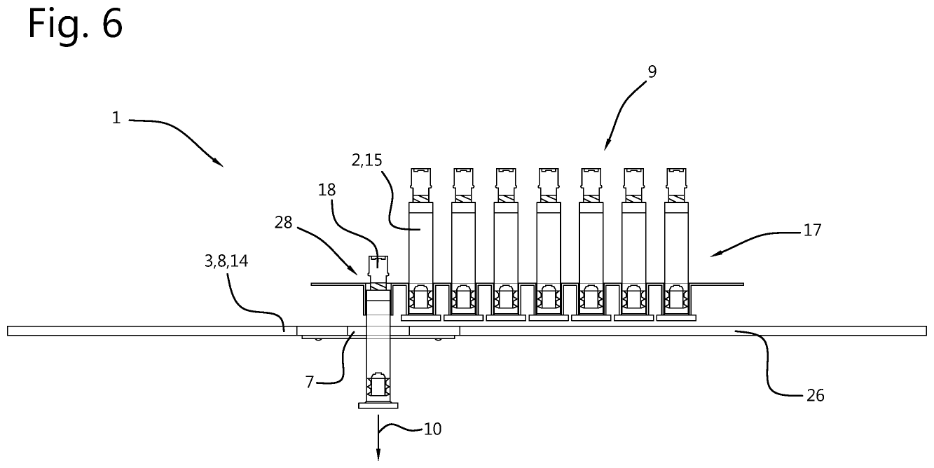

Generally speaking, the invention also provides a method for inspecting a single medication container 2 using an aforementioned medication container inspection device 1 or a medication container inspection assembly 16, as for instance shown in FIGS. 1-2 and 4-5, comprising:
automatically transporting one or more medication containers 2 to the inspection station 4 with the transport system 10,
inspecting a single medication container 2 for container defects with the inspection system 5 comprising the inspection apparatus 6, wherein the inspection system 5 performs multiple inspecting steps in the inspection station 4, and
entangling or agitating the single medication container 2 to increase the visibility of container defects while being inspected by the inspection apparatus 6. FIG. 6 shows a schematic view of an example embodiment of a method for feeding a syringe 15 from a syringe nest 17 to the transport system 10 via an infeed opening 7 with the injection end 28 upwards. Normally, syringes 15 are supplied in a nest 17 and a tub. The syringes 15 are suspended in the tub, in a nest 17 with the injection end 28 (for instance comprising a syringe cap 18) downwards. The nest 17 and syringes 15 are then removed from the tub with a so-called ‘flipping tool’. With the flipping tool, the nest 17 can be rotated without the syringes 15 falling out. The nest 17 is now rotated and the syringes 15 can now stand (are supported) on the infeed table 26, as shown in FIG. 6. After rotating the nest 17, the nest can slide over the infeed table to make sure that every syringe can be moved to one of the infeed openings. The syringes 15 can now be lowered one by one through the infeed opening 7, as shown in FIG. 6. The transport system 10 may then “take over” to transport the syringes 15 to the inspection station 4 (not shown in FIG. 6).

### LIST OF REFERENCE NUMERALS

1. Medication container inspection device
2. Medication container
3. Housing
4. Inspection station
5. Inspection system
6. Inspection apparatus
7. Infeed opening (7′, 7″)
8. Side of the housing
9. Infeed
10. Transport system/engagement device (10′, 10″)
11. Engagement position
12. Viewing device
13. Illumination device
14. Top side of the housing
15. Syringe
16. Medication container inspection assembly
17. Nest
18. Syringe cap
19. Outfeed
20. Outfeed opening
21. Mechanical link
22. Nest platform
23. Joint
24. Lid
25. Rail
26. Infeed table
27. Manipulator
28. Injection end

1. Medication container inspection device for inspecting one or more medication containers, comprising:
an inspection station with an inspection system comprising an inspection apparatus configured for inspecting a single medication container for container defects, and a manipulator to entangle or agitate the single medication container to increase the visibility of container defects while being inspected by the inspection apparatus,
a transport system for automatically transporting one or more medication containers, such as a single medication container, to the inspection station, wherein the one or more medication containers can be fed to the transport system with an infeed,
wherein the inspection system is configured to perform multiple inspecting steps in the inspection station.

2. Container inspection device according to claim 1, comprising:
a housing, wherein the inspection station is arranged in the housing, with an infeed opening arranged at a side of the housing, for allowing an infeed to feed one or more medication containers to the infeed opening, wherein the transport system is arranged for automatically transporting one or more medication containers to the inspection station from the infeed opening.

3. Container inspection device according to claim 1, wherein the manipulator is configured for engaging the single medication container at a plurality of engagement positions on the single medication container.

4. Container inspection device according to claim 1, wherein the inspection system is configured to perform all of the multiple inspecting steps in the inspection station.

5. Container inspection device according to claim 1, wherein the inspection apparatus comprises one or more viewing devices for viewing the single medication container in the inspection station.

6. Container inspection device according to claim 5, wherein the inspection system comprises one or more illumination devices for illuminating the single medication container in the inspection station when the single medication container is viewed by the one or more viewing devices.

7. Container inspection device according to claim 2, wherein the side of the housing comprising the infeed opening is the, during use, top side of the housing.

8. Container inspection device according to claim 7, wherein the infeed opening is arranged vertically above the inspection station, such that the transport system can transport the one or more medication containers vertically from the infeed opening to the inspection station.

9. Container inspection device according to claim 1, wherein the manipulator doubles as the transport system for automatically transporting the one or more medication containers to the inspection station or the transport system doubles as the manipulator to entangle or agitate the one or more medication containers.

10. Container inspection device according to claim 1, wherein the single medication container is at least partly transparent, such that the contents of the single medication container can be viewed from outside the container.

11. Container inspection device according to claim 1, wherein the single medication container comprises a fluid and/or a solid and/or the single medication container comprises a syringe, vial or ampoule.

12. (canceled)

13. Container inspection device according to claim 1, comprising:
multiple inspection stations each with an inspection system comprising an inspection apparatus, configured for inspecting a single medication container for container defects, and a manipulator to entangle or agitate the single medication container to increase the visibility of container defects while being inspected by the inspection apparatus, wherein at least one of the inspection stations is configured to perform multiple inspecting steps in the inspection station, and
a transport system for automatically transporting one or more medication containers to each of the inspection stations, wherein the one or more medication containers can be fed to the transport system with an infeed.

14. Medication container inspection assembly, comprising:
a medication container inspection device according to claim 1; and
an infeed for feeding one or more medication containers to the transport system.

15. Container inspection assembly according to claim 14, wherein, the infeed is arranged at a side of the housing for feeding one or more medication containers to an infeed opening.

16. Container inspection assembly according to claim 15, wherein the infeed holds vials, ampoules, syringes or a nest of syringes.

17. Container inspection assembly according to claim 16, wherein, the infeed is configured for feeding the syringes, vials or ampoules to the infeed opening or, if applicable, the syringes to the infeed opening from the nest.

18. Container inspection assembly according to claim 16, wherein the nest comprises syringes with injection ends for instance comprising syringe caps wherein the syringes are arranged in the nest with the injection ends upwards, wherein the syringes are fed to the transport system with the injection ends upwards.

19. (canceled)

20. Container inspection assembly according to claim 18, wherein the transport system is arranged for automatically transporting syringes to the inspection station with the injection ends upwards.

21. (canceled)

22. Container inspection assembly according to claim 14, comprising an outfeed for transporting a single medication container away from the inspection station.

23. Method for inspecting a single medication container using a medication container inspection device according to claim 1, comprising:
automatically transporting one or more medication containers to the inspection station with the transport system,
inspecting a single medication container for container defects with the inspection system comprising the inspection apparatus wherein the inspection system performs multiple inspecting steps in the inspection station, and
entangling or agitating the single medication container to increase the visibility of container defects while being inspected by the inspection apparatus.

24. (canceled)
